(19) United States
(12) Patent Application Publication
(10) Pub. No.: US 2015/0374725 A1
(43) Pub. Date: Dec. 31, 2015

(54) METHODS AND COMPOSITIONS FOR TREATING AND PREVENTING INTESTINAL INJURY AND DISEASES RELATED TO TIGHT JUNCTION DYSFUNCTION

(71) Applicant: PRELIEF INC., Egg Harbor Township, NJ (US)

(72) Inventors: Alan E. KLIGERMAN, Egg Harbor Township, NJ (US); Margaret T. WEIS, Amarillo, TX (US)

(21) Appl. No.: 14/768,373

(22) PCT Filed: Feb. 20, 2014
(86) PCT No.: PCT/US14/17408

§ 371 Date: Aug. 17, 2015

Related U.S. Application Data
(60) Provisional application No. 61/766,976, filed on Feb. 20, 2013.
(60) Provisional application No. 61/897,672, filed on Oct. 30, 2013.
(60) Provisional application No. 61/827,080, filed on May 24, 2013.

(51) Int. Cl. A61K 31/661; A61K 45/06; A61K 9/50

(52) U.S. Cl. CPC A61K 31/661; A61K 9/50; A61K 45/06

(57) ABSTRACT

Methods and compositions for treating and preventing intestinal injury are described. In particular, methods and delayed-release enteric coated compositions of glycerophosphate salts for treating and preventing an intestinal injury, such as exercise-induced intestinal injury, intestinal injury induced or aggravated by ingestion of a nonsteroidal anti-inflammatory drug (NSAID), or exercise-induced intestinal injury aggravated by an NSAID, are described. Orally or nasally administering a therapeutically effective amount of a glycerophosphate salt, particularly calcium glycerophosphate, to a subject engaging in exercise, particularly a subject engaging in exercise and ingesting a NSAID for the treatment or prevention of musculoskeletal pain resulting from exercise, reduces exercise-induced intestinal injury, particularly exercise-induced intestinal injury aggravated by the NSAID. A method of increasing tight junction integrity in a subject in need thereof is also described. The method involves administering to the subject a composition comprising an effective amount of glycerophosphate, such as calcium glycerophosphate. A method of treating or preventing a disease related to tight junction dysfunction, such as an inflammatory disease or a cancer, is also described.

### CROSS-REFERENCE TO RELATED APPLICATIONS

This application claims priority to U.S. Patent Application Ser. No. 61/766,976, filed Feb. 20, 2013, U.S. Patent Application Ser. No. 61/827,080, filed May 24, 2013, and U.S. Patent Application Ser. No. 61/897,672, filed Oct. 30, 2013, the disclosures of which are herein incorporated by reference.

### FIELD OF THE INVENTION

The invention relates to methods and compositions for treating and preventing intestinal injury. In particular, the invention provides a method of reducing, alleviating, or preventing intestinal injury in a subject, such as that induced by exercise or induced or aggravated by the ingestion of a nonsteroidal anti-inflammatory drug, using a glycerophosphate salt. The invention also relates to methods and compositions for regulating tight junctions and for preventing or treating diseases related to tight junction dysfunction.

### BACKGROUND OF THE INVENTION

Blood flow distribution in the human and animal body is a function of oxygen demand, which in turn is dependent on activity. That is, flow is differentially distributed to those vascular beds serving organs with the highest oxygen demand. This is accomplished by constricting the vessels supplying low demand organs and relaxing those supplying high demand organs.

Blood flow exerts shear force on the vessel wall, stimulating the synthesis of cytoprotective agents such as nitric oxide and prostaglandins. Interruptions or reduction of flow reduces shear force, reducing the synthesis of these agents, and setting tissue up for ischemic damage (i.e., damage due to reduced or restricted blood flow).

The splanchnic circulation refers to the circulation of the blood to organs of the gastrointestinal tract, including the stomach, small intestine, colon, pancreas, liver, and spleen. The splanchnic flow (blood flow to the gut) is highly variable, and when blood flow to the gut is reduced, the gut may be operating under conditions of mild to moderate ischemia.

In particular, the splanchnic flow is curtailed during heavy exercise because blood flow is diverted to skeletal muscle, heart, and skin. Thus, during vigorous exercise the gut often operates under conditions of mild to moderate ischemia. This effect is greatly enhanced by concomitant ingestion of ibuprofen, a non-steroidal anti-inflammatory agent or NSAID (see van Wijk et al., Medicine & Science in Sports & Exercise 44, 2257-2262 (2012); hereinafter referred to as “van Wijk”).

NSAIDs block the synthesis of prostaglandins, substances that mediate pain and inflammation. However, prostaglandins are also cytoprotective, particularly to the gastrointestinal (GI) tract. Consequently, the chronic use of NSAIDs is associated with adverse GI effects such as ulcers and compromised intestinal barrier function and, as a result, increased gut permeability (E. Focalin, Ann. Clan. Lab. Sci. 2, 67-81 (1998)).

Athletes routinely seek ways to prevent exercise-induced physical pain, subsequently improving their physical performance. Commonly, athletes use NSAIDs before, during and/or after physical activity to treat existing musculoskeletal pain, or in anticipation of musculoskeletal pain associated with, or induced by, exercise and physical activity. Ingestion of NSAIDs alone can cause intestinal injury. However, when taken in combination with exercise, the effect of the NSAID on intestinal injury, particularly in compromised intestinal barrier function and increased intestinal permeability, is greatly enhanced.

Intestinal fatty acid binding protein (I-FABP) is a 15 kDa protein whose appearance in the plasma is a marker of enterocyte (intestinal absorptive cells) damage. Recently, van Wijk reported that ibuprofen aggravates exercise-induced small intestinal injury, specifically increasing gastroduodenal and small intestinal permeability, when administered to individuals engaging in moderate exercise. This study measured the appearance of I-FABP in the peripheral plasma before, during, and after a defined exercise session (cycling). When 400 mg of ibuprofen were administered to subjects before exercise, the plasma I-FABP levels were significantly elevated relative to the plasma I-FABP levels measured from just exercise alone. The subjects also experienced a significant increase in intestinal permeability, as assessed by measuring the uptake of non-metabolizable mono- and disaccharides that are ordinarily excluded by the intestinal barrier. In summary, both exercise and ibuprofen increased gut permeability. The effect of the two combined was greater than the sum of the individual effects, suggesting that the two act via different pathways, both converging on enterocyte viability and enterocyte-enterocyte barrier function.

NSAIDs are commonly used by the general population to alleviate minor aches and pains and to reduce fever and inflammation. In addition, despite the risks, NSAIDs are commonly used by athletes to treat existing musculoskeletal pain and prevent any anticipated musculoskeletal pain associated with, or induced by, exercise. For particular sports, usage among participants has been reported to be as high as 90% (T. Gorski, et al., Br. J. Sports Med. 45(2), 85-90 (2011); E. Taioli, Br. J. Sports Med. 41(7), 439-441 (2007); W. V. Thuyne, Clin. J. Sport Med. 18(2), 143-147 (2008).)

Spaces between epithelial or endothelial cells must be tightly sealed so that molecules transported from one layer to another cannot diffuse back through these spaces. Tight junctions create a barrier in epithelial and endothelial cells, regulating the movement of water and solutes through paracellular spaces. Tight junctions also function to maintain cell polarity by forming a fence to prevent intermixing of molecules in the apical membrane with those in the lateral membrane. In many diseases, such as cancers or inflammatory diseases, tight junction dysfunction occurs. (See e.g., C. Foster; Histochem Cell Biol.; 130(1): 55-70 (July 2008).

It was reported that sphingosine-1-phosphate (S1P), a serum-borne bioactive lipid, activates tight-junction-associated protein Zonula Occludens-1 (ZO-1), which in turn plays a critical role in regulating endothelial chemotaxis and barrier integrity. (See Lee et al., J Biol Chem. 29; 281(39):29190-200 (September 2006); Epub Aug. 6, 2006.)

It is believed that NSAIDS likely act by two mechanisms to reduce the “tightness” of enterocyte tight junctions, consequently leading to increased intestinal permeability. Some NSAIDS sold over-the-counter in the US have the side effect of inhibiting oxidative phosphorylation. Consequently, the NSAIDS interfere with the production of cellular energy. In that respect, they would be expected to exacerbate the effect of reduced blood flow to the gut observed during exercise. The reduced flow impairs the availability of oxygen, while the NSAIDs simultaneously reduce the ability of the cell to use whatever oxygen is available.

Recent evidence shows that in cultured enterocytes, S1P not only governs expression of specific tight junction proteins, but also increases the “tightness” of existing tight junctions (Greenspon et al., Dig. Dis. Sci. 56, 1342-1353 (2011)). The effect on junctional integrity could be observed in as little as 30 minutes following exposure to S1P. S1P is created by the phosphorylation of the cellular sphingolipid, sphingosine. It is only in its phosphorylated form, as S1P, that the important governing, “signaling” effects are set into motion. The phosphorylated state of S1P is, however, under constant down-regulating activity by contiguous de-phosphorylating phosphatases.

There exists a need in the art for a composition and method to treat and prevent intestinal injury, and in particular, intestinal injury caused by ingestion of an NSAID, or induced by exercise. Even more particularly, there exists a need for a composition and method to treat and prevent exercise-induced intestinal injury for those who frequently consume NSAIDs prior to, during, or immediately following exercise to reduce and/or prevent exercise-associated stiffness or pain. Preferably such a method is non-toxic, non-hazardous, and without significant adverse side effects. There is also a need for novel methods and compositions for regulating tight junctions, which can be used for preventing or treating diseases related to tight junction dysfunction.

### BRIEF SUMMARY OF THE INVENTION

The present invention provides methods and compositions for treating or preventing intestinal injury or symptom thereof, including intestinal injury induced or aggravated by ingestion of a nonsteroidal anti-inflammatory drug (NSAID), exercise-induced intestinal injury, and exercise-induced intestinal injury aggravated by ingestion of an NSAID to treat or prevent musculoskeletal pain associated with exercising. According to embodiments of the present invention, administration of a glycerophosphate salt, and particularly administration of calcium glycerophosphate, to a subject is an effective method for reducing, alleviating, and/or preventing intestinal injury or symptom thereof in the subject.

In one general aspect, the present invention relates to a method of treating or preventing an intestinal injury or symptom thereof in a subject, the method comprising administering to the subject a composition comprising a therapeutically effective amount of a glycerophosphate salt such that intestinal injury or symptom thereof is treated or prevented. In one embodiment, the internal injury or symptom thereof is exercise-induced and the subject engages in exercise. Preferably, the composition is administered orally or nasally, more preferably orally.

In another general aspect, the present invention relates to a method of treating or preventing an intestinal injury or symptom thereof aggravated by a nonsteroidal anti-inflammatory drug (NSAID) in a subject, wherein the subject is administered the NSAID, the method comprising administering to the subject a composition comprising a therapeutically effective amount of a glycerophosphate salt such that the intestinal injury or symptom thereof aggravated by the NSAID is treated or prevented. Preferably, the composition is administered orally or nasally, more preferably orally. In one embodiment, the internal injury or symptom thereof is exercise-induced and the subject engages in exercise.

According to embodiments of the present invention, administering a therapeutically effective amount of a glycerophosphate salt, and preferably calcium glycerophosphate, to a subject results in the reduction, alleviation, or prevention of intestinal injury and/or one or more symptoms thereof, such as those that are exercise-induced, as compared to the intestinal injury and/or symptoms thereof that result when the subject is not administered a glycerophosphate salt. The glycerophosphate salt, and preferably calcium glycerophosphate, is also effective in reducing, alleviating or preventing intestinal injury and/or one or more symptoms thereof further aggravated by ingestion of an NSAID, such as those that are exercise-induced, as compared to the intestinal injury and/or symptoms thereof that may result when the subject is not administered the glycerophosphate salt.

The present invention also relates to a novel enteric coated composition comprising a therapeutically effective amount of a glycerophosphate salt, and methods of treating or preventing intestinal injury in a subject, increasing tight junction integrity in a subject, and treating or preventing diseases related to tight junction dysfunction in a subject, comprising administering to the subject an enteric coated composition according to the invention, such as a delivery site-specific composition.

In one general aspect, the present invention provides an enteric coated composition comprising a therapeutically effective amount of a glycerophosphate salt. According to embodiments of the present invention, an enteric coated composition comprises:

a core comprising a therapeutically effective amount of a glycerophosphate salt; and an enteric coating surrounding the core,

In a preferred embodiment, the enteric coating prevents release of the glycerophosphate salt from the core until the composition is assured of substantially reaching at least the jejunal/ileal sectors of the small intestine, as well as the large intestine, of the subject.

In another general aspect, the present invention also relates to method of increasing tight junction integrity in a subject in need thereof, comprising administering to the subject a composition comprising an effective amount of glycerophosphate.

In another general aspect, the invention also relates to methods of treating or preventing a disease related to tight junction dysfunction in a subject in need thereof, comprising administering to the subject a composition comprising an effective amount of glycerophosphate.

### BRIEF DESCRIPTION OF THE DRAWINGS

The following detailed description of the invention will be better understood when read in conjunction with the appended drawings. For the purpose of illustrating the invention, there are shown in the drawings embodiments which are presently preferred. It should be understood, however, that the invention is not limited to the embodiments shown. In the drawings:

FIG. 1 is a graph of baseline mannitol flux versus time in the presence and absence of increasing concentrations of CGP;

FIG. 2 are graphs showing the effect of CGP on trans-epithelial electrical resistance (TEER) in the presence and absence of increasing concentrations of CGP;

FIG. 3 is a graph showing the effect of CGP on baseline production of sphingosine 1-phosphate in Caco-2 cells;

FIG. 4 is a graph showing the increase in mannitol penetration in Caco-2 cells four hours after the administration of calcium glycerophosphate (CGP) to the cells;

FIG. 5 are graphs of transepithelial mannitol flux during hypoxia;

FIG. 6 are graphs of TEER during hypoxia;

FIG. 7 are graphs showing the effect of CGP on transepithelial mannitol flux induced by cytomix; and

FIG. 8 are graphs showing the effect of CGP on transepithelial electrical resistance during cytomix ((TNF-α), (IFN-γ), and (IL-β)) treatment.

### DETAILED DESCRIPTION OF THE INVENTION

Unless defined otherwise, all technical and scientific terms used herein have the same meaning as is commonly understood by one of ordinary skill in the art to which this invention pertains. All publications and patents referred to herein are incorporated by reference. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

It must be noted that as used herein and in the appended claims, the singular forms “a,” “an,” and “the” include plural reference unless the context clearly dictates otherwise.

As used herein, the term “glycerophosphate salt” refers to any salt comprising a glycerophosphate moiety and a non-toxic cation. Non-limiting examples of non-toxic cations include calcium, sodium, potassium, and magnesium. Glycerophosphate salts suitable for use in the present invention include, but are not limited, to calcium glycerophosphate, sodium glycerophosphate, potassium glycerophosphate, and magnesium glycerophosphate. Preferably, the glycerophosphate salt is calcium glycerophosphate.

As used herein, the term “calcium glycerophosphate” or “CGP” refers to a compound having a molecular formula of C₃H₇CaO₆P and a molecular weight of 210.04 in anhydrous form. “CGP” can also exist as a hydrate, including the monohydrate and dihydrate forms. “CGP” is also known as 1,2,3-propanetriol, mono(dihydrogen phosphate) calcium salt (1:1), calcium glycerinophosphate, calcium phosphoglycerate and NEUROSIN®. Three CGP isomers exist, namely β-glycerophosphoric acid calcium salt ((HOCH₂)₂CHOPO₃Ca) and D(+) and L(−)-a-glycerophosphoric acid calcium salt (HOCH₂CH(OH)CH₂OPO₃Ca). Any one isomer, or any combination of two or more isomers, may be used as the CGP according to this invention.

CGP can be synthesized using methods known in the art. CGP can also be obtained from various commercial sources. The commercially available CGP preparations include, but are not limited to, those available from AkPharma Inc. (Pleasantville, N.J. 08232), Astha Laboratories Pvt, Ltd, (B-4, Industrial Estate, Sanathnagar, Hyderabad-18, India), Seppic Inc. (30 Two Bridges Road, Fairfield, N.J. 07004), and numerous other manufacturers and distributors around the world.

As used herein, the term “nonsteroidal anti-inflammatory drug” or “NSAID” refers to a class of compounds that have analgesic (pain reducing), antipyretic (fever reducing), and/or anti-inflammatory effects. Examples of NSAIDs include, but are not limited to, ibuprofen, aspirin, naproxen, fenoprofen, ketoprofen, flurbiprofen, oxaprozin, indomethacin, sulindac, etodolac, diclofenac, piroxicam, meloxicam, tenoxicam, droxicam, lornoxicam, and isoxicam, and pharmaceutically acceptable salts thereof.

As used herein, the term “exercise” refers to any form of physical activity. In one embodiment, exercise can refer to any activity aimed at improving flexibility, or the range of motion of muscles and joints, such as stretching. In another embodiment, exercise can refer to any aerobic activity aimed at increasing cardiovascular endurance, including, but not limited to, cycling, swimming, walking, rowing, running, hiking, and tennis. In yet another embodiment, exercise can refer to any anaerobic activity aimed at increasing short-term muscle strength, such as, for example, weight training, interval training, tennis, football at all age and competitive levels, soccer, sprinting, or any other physically demanding sport or activity. Although a method of the present invention can treat intestinal injury caused by any form of exercise in view of the present disclosure, intestinal injury may be more likely to occur following aerobic exercise as compared to anaerobic exercise because the oxygen demand in aerobic exercise is greater, and thus the need for treatment following aerobic exercise may also be greater.

As used herein, the term “subject” means any animal, preferably a mammal, most preferably a human, to whom will be or has been administered compositions or compounds according to embodiments of the present invention. Examples of mammals include, but are not limited to, cows, horses (most particularly race horses), sheep, pigs, cats, dogs, mice, rats, cats, dogs, rabbits, guinea pigs, monkeys, humans, etc., most preferably humans.

As used herein, the term “intestine” refers to the intestinal tract. The intestinal tract includes the small intestine and the large intestine. The small intestine is composed of the duodenum (i.e., the uppermost portion proximal to the stomach), the jejunum (i.e. the middle portion/segment), and the ileum (i.e., the final section prior to the large intestine). As used herein, “junction between the duodenum and jejunum portions” refers to the portion of the small intestine wherein the duodenum segment meets the jejunum segment. As used herein, “junction between the duodenum and jejunum portions” refers to the portion of the small intestine wherein the duodenum segment meets the jejunum segment.

The intestinal barrier to intestinal cell permeability is dependent on the formation of tight junctions between adjacent enterocytes, i.e., intestinal cells. Tight junctions are composed of strands of proteins (principally claudins and occludins) that girdle the cell near the intestinal lumen. The junctional strands of one cell bind to those on an adjacent cell, forming a seal between the cells, in much the same way that tongue-and-groove joints hold floorboards together. The “tightness” of the tight junction between cells is a direct function of the number of strands, and can be influenced by the oxidative state of the cell as well as soluble factors on the surface of the cell.

Substances move across the intestinal barrier by either the transcellular (across the cell) or paracellular (between the cells) routes. By definition, substances that travel between cells are able to traverse tight junctions. The rate at which they do so depends on both the molecular weight of the substance and the “tightness” of the tight junctions.

As used herein, the term “intestinal injury” refers to damage to the intestine, and in particular damage to the enterocytes (intestinal cells) themselves. Enterocytes are the predominant cells in the small and large intestinal mucosa responsible for the final digestion and absorption of nutrients, electrolytes, and water. “Intestinal injury” also refers to damage to the junctions between intestinal cells, i.e., the enterocyte-enterocyte tight junctions, such that normal barrier intestinal function is impaired, diminished, or has deteriorated. The scope of the term “intestinal injury” as used herein is intended to further encompass increased intestinal permeability as a result of compromised enterocyte-enterocyte tight junctions, consequently compromising the intestinal epithelial barrier integrity and permitting diffusion of substances across the intestinal barrier that would ordinarily be excluded. “Intestinal injury” as used herein also refers to symptoms, and the onset of symptoms, of intestinal injury, including, but not limited to, epigastric pain, flatulence, dyspepsia (indigestion), and belching.

As used herein, the terms “increased gut permeability” and “increased intestinal permeability” refer to a decrease in the “tightness” of the tight junctions between intestinal cells.

As used herein, a “therapeutically effective amount” means an amount of a glycerophosphate salt that is effective in reducing, alleviating, causing intestinal injury to develop to a lesser extent, or preventing an intestinal injury. A “therapeutically effective amount” is also an amount of a glycerophosphate salt that is effective in reducing, alleviating, causing to develop to a lesser extent, or preventing any symptoms associated with, or indicative of, an intestinal injury.

As used herein, the terms “treating,” “treat,” or “treatment” refer to the administration of an amount of a glycerophosphate salt to a subject that is effective in reducing, alleviating, or preventing intestinal injury and/or one or more symptoms associated with intestinal injury in a subject.

As used herein the terms “preventing,” “prevent,” or “prevention” refer to the administration of a therapeutically effective amount of a glycerophosphate salt to a subject before the onset of symptoms associated with intestinal injury, such that any intestinal injury, or associated symptoms, will be prevented altogether, will be time-delayed as to occurrence, or may still occur, but will do so to a lesser extent than in the absence of a glycerophosphate salt.

Embodiments of the present invention comprise methods of treating or preventing intestinal injury in a subject. Because increased intestinal permeability can be the consequence of exercise-induced blood flow diversion during exercise from the splanchnic circulation (i.e. blood flow to the gut) to meet increased musculoskeletal oxygen demand, exercise alone can cause intestinal injury. The intestinal injury induced by exercise can be further aggravated or exacerbated by ingestion of an NSAID. Thus, embodiments of the present invention also comprise methods of treating or preventing exercise-induced intestinal injury in a subject, methods of treating or preventing intestinal injury aggravated by an NSAID in a subject, and methods of treating or preventing exercise-induced intestinal injury aggravated by the ingestion of an NSAID in a subject.

As used herein the phrase “exercise-induced intestinal injury” refers to intestinal injury, or symptoms of intestinal injury, caused by exercise such that the resulting intestinal injury or symptoms of intestinal injury are greater than the intestinal injury, or symptoms of intestinal injury that would have resulted in the absence of exercise. As used herein, the phrase “exercise-induced intestinal injury aggravated by an NSAID” refers to intestinal injury, or symptoms of intestinal injury, caused by exercise and exacerbated by, or increased by, ingestion of an NSAID, such that the resulting intestinal injury, or symptoms of intestinal injury, are greater than the intestinal injury, or symptoms of intestinal injury, that would have resulted in the absence of exercise and the ingestion of an NSAID.

### Methods of Treating or Preventing Intestinal Injury or Symptoms

According to embodiments of the present invention, a method of treating or preventing intestinal injury or a symptom thereof in a subject comprises administering to the subject a therapeutically effective amount of a glycerophosphate salt. In one embodiment, the intestinal injury or symptom thereof to be treated or prevented is exercise-induced. In another embodiment, the intestinal injury or symptom thereof to be treated or prevented is exercise-induced and further aggravated by ingestion of an NSAID.

Thus, according to embodiments of the present invention, a subject to be administered an effective amount of a glycerophosphate salt can be one who is engaging in exercise, such that the intestinal injury to be treated or prevented is exercise induced. A subject to be administered an effective amount of a glycerophosphate salt can also be one who ingests an NSAID, such that the intestinal injury to be treated or prevented is induced by the NSAID. A subject to be administered a glycerophosphate salt according to the invention can further be one who is engaging in exercise and ingesting an NSAID, such as to alleviate current or anticipated exercise-associated musculoskeletal pain. Non-limiting examples of exercise-associated musculoskeletal pain that can be treated by the intake of an NSAID include inflammation of the joints, such as in knees or elbows; joint injuries, such as sprains; inflammation of the tendons (tendonitis); strained or torn ligaments, such as a torn anterior cruciate ligament (ACL); tibial stress syndrome (shin splints); plantar fasciitis; and strained hamstring, quadriceps, gluteal, bicep, or tricep muscles (pulled muscles). Exercise-associated musculoskeletal pain that can be treated by intake of an NSAID also includes simple muscle and joint aches post-exercise, with no specific trauma experienced or indicated. For the purposes of this disclosure, the actual effectiveness of the NSAID in producing relief of the perceived or anticipated pain at any locale, is not of immediate concern; only the fact that the exerciser ingest such NSAID at or near the time of such exercise, putting the intestinal cells at specific risk, thereby.

According to the present invention, a subject's ingestion of an NSAID and engagement in exercise need not be simultaneous, but only contemporaneous or concurrent, such that an effective amount of an NSAID is present in the subject in imminent anticipation of, or at the initiation of exercise, and thus has the potential to exacerbate or aggravate any intestinal injury induced by the exercise itself from the moment of initiation of the exercise. For example, an NSAID, such as ibuprofen, can be ingested 60 minutes prior to engaging in exercise, such as running.

In one embodiment of the present invention, the administration of a therapeutically effective amount of a glycerophosphate salt results in a clinically observable beneficial effect in the reduction, alleviation, or prevention of one or more symptoms of intestinal injury, such that symptoms are about 90%, 80%, 70%, 60%, 50%, 40%, 30%, 20%, 10% or less than the symptoms that would have resulted from intestinal injury, had the subject not received a therapeutically effective amount of the glycerophosphate salt.

In another embodiment of the present invention, the administration of a therapeutically effective amount of a glycerophosphate salt results in a clinically observable beneficial effect in the reduction, alleviation, or prevention of one or more symptoms of intestinal injury aggravated by an NSAID, such that symptoms are about 90%, 80%, 70%, 60%, 50%, 40%, 30%, 20%, 10% or less than the symptoms that would have resulted from ingestion of an NSAID, had the subject not received a therapeutically effective amount of the glycerophosphate salt.

In yet another embodiment of the present invention, the administration of a therapeutically effective amount of a glycerophosphate salt results in a clinically observable beneficial effect in the reduction, alleviation, or prevention of one or more symptoms of exercise-induced intestinal injury, such that symptoms are about 90%, 80%, 70%, 60%, 50%, 40%, 30%, 20%, 10% or less than the symptoms that would have resulted from exercise alone, had the subject not received a therapeutically effective amount of the glycerophosphate salt.

And in yet another embodiment of the present invention, the administration of a therapeutically effective amount of a glycerophosphate salt results in a clinically observable beneficial effect in the reduction, alleviation, or prevention of one or more symptoms of exercise-induced intestinal injury aggravated by an NSAID, such that symptoms are about 90%, 80%, 70%, 60%, 50%, 40%, 30%, 20%, 10% or less than the symptoms that would have resulted from exercise and ingestion of an NSAID, had the subject not received a therapeutically effective amount of the glycerophosphate salt.

In yet another embodiment of the present invention, the clinically observable beneficial effect can be a situation wherein intestinal injury, intestinal injury aggravated by an NSAID, exercise-induced intestinal injury or exercise-induced intestinal injury aggravated by an NSAID, and/or one or more symptoms thereof, is prevented from further aggravation or development, or subsequently develops to a lesser degree, than would have resulted had the subject not received a therapeutically effective amount of a glycerophosphate salt.

In yet another embodiment of the present invention, the clinically observable beneficial effect can be a situation where intestinal injury, intestinal injury aggravated by an NSAID, exercise-induced intestinal injury or exercise-induced intestinal injury aggravated by an NSAID, and/or one or more symptoms thereof, is prevented from occurring, such that the subject does not experience any intestinal injury or symptoms of intestinal injury that would have resulted had the subject not received a therapeutically effective amount of a glycerophosphate salt.

In a preferred embodiment, the glycerophosphate salt administered to the subject is calcium glycerophosphate.

In view of the present disclosure, standard procedures can be performed to evaluate the effect of administration of a glycerophosphate salt to a subject, thus allowing a skilled artisan to determine the effective amount of the glycerophosphate to be administered to a subject. The clinically beneficial effect of the glycerophosphate salt can be readily apparent to the subject. For example, a subject administered an effective amount of a glycerophosphate salt who also ingested an NSAID prior to exercising can subsequently experience no indigestion, epigastric pain, or flatulence during and after exercise as compared to the amount of indigestion, epigastric pain, or flatulence experienced had the glycerophosphate salt not been administered. Symptoms directly observable to the subject can also include constipation or diarrhea or symptoms of inflammation, such as achy joints, muscle tenderness, etc. The symptoms to be evaluated can also resemble the musculoskeletal symptoms normally anticipated following vigorous exercise, such as muscle or joint pain etc.

The clinically beneficial effect of a glycerophosphate salt can also be determined by a clinical assay to evaluate the efficacy of the glycerophosphate salt in preserving intestinal barrier integrity, and thus treating or preventing intestinal injury. In this case, the clinically beneficial effect can be determined objectively, and the clinically beneficial effect may not necessarily be directly observable or readily apparent to the subject. For example, enterocyte damage, such as that which might result from impaired oxygen availability and/or utilization, liberates intestinal fatty acid binding protein (I-FABP) from the cell. I-FABP then enters circulation, and can be detected in plasma. Thus, plasma I-FABP is a marker of intestinal cell damage and I-FABP levels can be readily measured, such as by enzyme linked immunoabsorbent assay (ELISA), to determine the effect of the glycerophosphate salt on reducing intestinal injury. Another illustrative example of a clinical assay for evaluating intestinal injury and the efficacy of a glycerophosphate salt in preventing or reducing intestinal injury is a multiple sugar assay as described in J. Chromatog. B Analyt. Tech. Biomed. Life Sci., 879(26), 2794-2801 (2011), incorporated herein by reference. The multiple sugar assay measures intestinal cell permeability by measuring the uptake and urinary excretion of non-metabolizable mono- and disaccharides. These sugars are taken up by paracellular (between cells) routes, but only when the tightness of the enterocyte-enterocyte junctions is reduced.

Dosages of a glycerophosphate salt administered to a subject to provide a therapeutically effective amount of the glycerophosphate salt are not limited to a particular value. One of ordinary skill in the art will recognize that the therapeutically effective amount of a glycerophosphate salt necessary to observe a clinically beneficial effect will depend on additional factors such as the weight, age, gender, etc. of a subject to be treated, as well as their intestinal health to begin with, and will be readily able to determine the appropriate dosage that provides a therapeutically effective amount. Additional factors to be considered in determining the dosing regimen and therapeutically effective amount of a glycerophosphate salt administered to a subject according to the present invention include, but are not limited to, the sensitivity of the subject to NSAIDs and the intensity and regularity of exercise engaged in by the subject. For example, to treat or prevent exercise induced intestinal injury aggravated by ingestion of an NSAID in a subject, the subject can be administered CGP. Dosage of CGP relative to an NSAID can cover a wide range, and preferably CGP is administered in a mole ratio of CGP:NSAID ranging from 0.1:1 to 10:1. As an illustrative example, if the NSAID to be used is ibuprofen, a typical dosage would be a 1:1 ratio of CGP:ibuprofen by weight, in view of the fact that CGP and ibuprofen have nearly the same molecular weight (MW CGP=210.04; MW ibuprofen=206.29 g/mol). In this case, 200 mg of CGP can be administered for a 200 mg dose of ibuprofen. Likewise, 400 mg of CGP can be administered for a 400 mg dose of ibuprofen, etc. If aspirin (MW=189 g/mol) is administered as the NSAID, where the recommended dosage of aspirin can be 650 mg, the corresponding dosage of CGP can be 800 mg. For other NSAIDs, the dose of CGP would be similarly matched with the recommended or typical dose of the NSAID. When administered to a subject to treat or prevent exercise-induced intestinal injury in the absence of an NSAID, the dose of CGP, or other glycerophosphate salt, can be the same as that which would be administered had an NSAID also been ingested.

The glycerophosphate salt and NSAID can be administered in a single composition, such as a combination tablet or capsule, or they can be administered in separate compositions, such as two separate tablets or capsules. In either case, both the glycerophosphate salt and NSAID need not be ingested simultaneously, but must be ingested contemporaneously or concurrently such that both the glycerophosphate salt and NSAID contact the vulnerable intestinal cells contemporaneously with one another.

According to one embodiment of the present invention, a therapeutically effective amount of a glycerophosphate salt is administered orally to a subject in solid form or liquid form, such as a powder or a liquid mixture. When administered orally as a solid, the glycerophosphate salt can be, for example, in the form of a powder, tablet, capsule, caplet, gelcap, or liquid gel. When administered orally as a liquid, the glycerophosphate salt can be, for example, in the form of an emulsion, solution, suspension, syrup, or elixir.

According to another embodiment of the present invention, a therapeutically effective amount of a glycerophosphate salt is administered nasally to a subject as a solid powder or liquid mixture in a nasal spray/inhalant mixture. When administered nasally as a nasal spray/inhalant mixture, the glycerophosphate salt can be in the form of particles, or as a liquid emulsion, solution syrup, or elixir dissolved/suspended in an aqueous carrier solution/suspension. In this embodiment, delayed-release is not necessary.

In a preferred embodiment, the glycerophosphate salt is administered orally. While oral administration is the preferred method because nasal administration is not as direct of a route to the intestines, owing to substantial absorption at the nasal membrane level, some of the nasally administered product can easily be swallowed via rear area upper respiratory drainage down through the esophagus, thereby being routed directly to the intestinal tract, although via such routing there is no delayed-release for that portion which proceeds gastrointestinally.

According to another embodiment of the present invention, the glycerophosphate salt can be administered orally in combination with a sports-directed food or drink. As used herein, the term “sports-directed food or drink” refers to any beverage or food item specifically marketed to athletes for enhancing performance of or recovery from, exercise, training, athletic performance, or any activity related to exercise or athletic performance. Non-limiting examples of sports-directed food and drink include energy bars, protein powders, protein shakes, and sports drinks, particularly those designed to aid in hydration after exercise or replace electrolytes. The glycerophosphate salt can be dissolved, or suspended in, a sports-directed drink and orally administered to a subject upon ingestion of the sports-directed drink by the subject. The glycerophosphate salt can also be combined with a sports-directed food and thus administered orally to a subject upon ingestion of the sports-directed food by the subject. For example, a CGP powder can be mixed with a protein powder, or disintegrated or suspended in a protein bar.

Compositions comprising a therapeutically effective amount of a glycerophosphate salt for use in the present invention can be formulated using any method known to those skilled in the art in view of the present disclosure. To prepare the compositions for administration to a subject, the glycerophosphate salt is mixed with a pharmaceutically acceptable carrier according to conventional pharmaceutical compounding techniques. Compositions for use in the present invention can comprise a therapeutically effective amount of a glycerophosphate salt that is about 10-90% glycerophosphate salt, preferably 50-90%, and most preferably 75-90% of the total weight of the composition. While the active portion of the composition preferably contains 100% of the glycerophosphate salt, the percentage of glycerophosphate in the entire formulated compound will be a function of the further weight of the protective coating involved to ensure that the active salts reaches the appropriate sections of the small and large intestines. The amount by weight of glycerophosphate salt will depend on the specific cation of the glycerophosphate salt. Preferably, the core comprises at least about 95% of the composition, with the protective coating comprising the balance. However, these percentages are not limiting and may be determined by routine experimentation.

Pharmaceutically acceptable carriers can include one or more excipients such as binders, suspending agents, emulsifying agents, wetting agents, lubricants, flavorants, sweeteners, preservatives, dyes, and coatings. Carriers can take a wide variety of forms depending on the form of preparation desired for administration. For liquid oral preparations, for example, suspensions, elixirs and solutions, suitable carriers and additives include water, glycols, oils, alcohols, flavoring agents, preservatives, coloring agents, and the like. For solid oral preparations, for example, powders, capsules, caplets, gelcaps and tablets, suitable carriers and additives include starches, sugars, diluents, granulating agents, lubricants, binders, disintegrating agents and the like. For nasal sprays/inhalant mixtures, the aqueous solution/suspension can comprise water, glycols, oils, emollients, stabilizers, wetting agents, preservatives, aromatics, flavors, and the like as suitable carriers and additives.

The solubility of CGP in water is approximately 1 gram in 100 mL. Thus, a solution/suspension of CGP can be prepared for oral administration according to the invention, for example, by dissolving/disintegrating and suspending CGP powder or tablets in water. Where a suspension is deemed acceptable, a concentration greater than 1% CGP can be used, and kept in suspension by use of any number of common, known stabilizers including, but not limited to, agar, sodium carboxylmethylcellulose gum, guar gum, etc. In a particular embodiment, CGP powder or tablets can be dissolved in a sports-directed drink, or disintegrated, suspended, or mixed into a sports-directed food. CGP can also be formulated in an extended release form suitable for several hours for use in the present invention. Methods are known to those skilled in the art to manufacture the extended release dosage form. A typical administration of a nasal spray could be from one spray per nostril daily to two sprays per nostril more than one time per day, as needed, depending on the amount of NSAID ingested by the subject. Compositions comprising other glycerophosphate salts, such as sodium, magnesium, or potassium glycerophosphate can be prepared similar to CGP compositions.

According to embodiments of the present invention, a glycerophosphate salt administered to treat or prevent intestinal injury can be administered to a subject at any time point. For example, a glycerophosphate salt can be administered once or twice daily to treat or prevent intestinal injury, or even more frequently, as desired, with no ill effects.

According to embodiments of the present invention, a glycerophosphate salt administered to treat or prevent intestinal injury aggravated by an NSAID can be administered to a subject at any point provided that the therapeutic effects of the glycerophosphate salt overlap with administration of the NSAID and its effects in aggravating intestinal injury, such that a clinically beneficial effect can be observed. The glycerophosphate salt can be administered, for example, simultaneously with the NSAID, immediately prior to ingestion of the NSAID, or immediately after ingestion of the NSAID.

According to embodiments of the present invention, a glycerophosphate salt administered to treat or prevent exercise-induced intestinal injury can be administered to a subject at any point provided that the therapeutic effects of the glycerophosphate salt overlap with the onset or persistence of exercise-induced intestinal injury, such that a clinically beneficial effect can be observed. Preferably, the glycerophosphate salt is administered immediately prior to the initiation of exercise, during exercise, or immediately following exercise.

According to embodiments of the present invention, a glycerophosphate salt administered to treat or prevent exercise-induced intestinal injury aggravated by an NSAID can be administered to a subject at any point provided that the therapeutic effects of the glycerophosphate salt overlap with administration of the NSAID and its effects in aggravating exercise-induced intestinal injury, such that a clinically beneficial effect can be observed. The glycerophosphate salt can be administered, for example, after ingestion of an NSAID but prior to the initiation of exercise, or the glycerophosphate salt can be administered after ingestion of an NSAID but during the course of exercise. The glycerophosphate salt can be administered prior to both the ingestion of an NSAID and exercise. The glycerophosphate salt can also be administered after ingestion of an NSAID, but after the completion of exercise, or simultaneously with the ingestion of an NSAID prior to the initiation of exercise.

Because glycerophosphate salts, and particularly CGP, are non-toxic, non-hazardous, and have no known side-effects, the number of times the glycerophosphate salt can be administered is not limited in any way. Thus, the glycerophosphate salt can also be administered multiple times per day, such as once, twice or three times or more per day. For example, when treating or preventing exercise-induced intestinal injury, a glycerophosphate salt, such as CGP, can be administered multiple times, such as before and after exercising, or before, during and after exercising. If the object is to prevent intestinal injury aggravated by an NSAID, a glycerophosphate salt can be administered each time an NSAID is ingested. When the object is to prevent any anticipated aggravated exercise-induced intestinal injury by an NSAID and associated symptoms thereof, it is preferable for the glycerophosphate salt to be administered prior to, or simultaneously with, ingestion of the NSAID. The situations presented above are intended to be non-limiting examples and it is to be understood that one of ordinary skill in the art will recognize when administration of CGP or other glycerophosphate salt, and particularly administration of CGP or other glycerophosphate salt relative to the ingestion of an NSAID, and even more particularly administration of CGP or other glycerophosphate salt relative to the ingestion of an NSAID and engagement in exercise, will yield a clinically beneficial effect according to the invention.

Compositions comprising a therapeutically effective amount of a glycerophosphate salt for use in the present invention can optionally comprise additional therapeutic additives, including NSAIDs, such as those discussed above. Thus, in a particular embodiment of the present invention, a glycerophosphate salt and an NSAID can be administered to a subject simultaneously as part of the same composition. Preferably, the glycerophosphate salt is CGP. A CGP and NSAID combination composition for use in the present invention preferably comprises an amount of CGP by weight that is about 10% to about 1000% of the weight of the NSAID in the mixture of CGP and NSAID, preferably 30% to 70%, more preferably 40% to 60%, and most preferably 50% of the weight of the NSAID in the composition. When taken simultaneously with an NSAID, but not part of a mutual composition with an NSAID, the weight of the CGP is preferably 1:1 relative to the weight of the NSAID. In a preferred embodiment, the NSAID is ibuprofen. As an illustrative but non-limiting example, a composition comprising CGP and ibuprofen can be administered to a subject 60 min prior to engaging in exercise, such as running, to treat and/or prevent exercise-induced intestinal injury according to the invention.

Individuals who chronically ingest NSAIDs prior to, during, or immediately following exercise to reduce exercise associated musculoskeletal pain can be athletes following strict training regimens, requiring intense exercise on an almost daily basis. For such subjects, extended release formulations of the glycerophosphate salt for the treatment or prevention of exercise-induced intestinal injury aggravated by NSAIDs can be desirable. Use of an extended release formulation of a glycerophosphate salt could limit the number of times administration of the glycerophosphate salt is required without sacrificing therapeutic efficacy. However, as discussed above, glycerophosphate salts, and particularly CGP, are non-toxic, non-hazardous, and have no known adverse side effects, thus subjects prescribed an extended release formulation of a glycerophosphate salt could optionally supplement their dosing regimens with standard glycerophosphate salt formulations to maximize the therapeutic effect. Preferably, an extended release or delayed release formulation for use in the present invention comprises CGP.

### Methods for Increasing Tight Junction Integrity and Treating or Preventing Diseases Related to Tight Junction Dysfunction

It is surprisingly discovered in the present invention that a glycerophosphate, such as calcium glycerophosphate (CGP), increases tight junction integrity and decreases transepithelial permeability.

Accordingly, a general aspect of the present invention relates to a method of increasing tight junction integrity in a subject in need thereof, comprising administering to the subject a composition comprising an effective amount of glycerophosphate, such as calcium glycerophosphate (CGP). Determination of the effective amount of glycerophosphate, the effectiveness of administration, the method of administration, and the components in the composition have been described previously with respect to method of treating intestinal injury or symptom thereof.

Another general aspect of the invention relates to a method of treating or preventing a disease related to tight junction dysfunction in a subject in need thereof. The method comprises administering to the subject a composition comprising an effective amount of glycerophosphate, such as calcium glycerophosphate (CGP). Determination of the effective amount of glycerophosphate, the effectiveness of administration, the method of administration, and the components in the composition have been described previously with respect to method of treating intestinal injury or symptom thereof.

Any disease related to tight junction dysfunction can be prevented or treated by the present method.

In one embodiment of the present invention, the disease related to tight junction dysfunction is a cancer, such as breast cancer, e.g., invasive ductal cancer; prostate cancer, e.g., prostatic adenocarcinomas; thyroid neoplasma, follicular adenoma; gastroesophageal reflux disease, e.g., Barrett's esophagus (dysplasia); lung cancer, e.g., basaloid squamous carcinoma; etc. (See C. Foster; Histochem Cell Biol.; 130(1): 55-70 (July 2008), the disclosures of which is incorporated by reference herein in its entirety.)

In another embodiment of the present invention, the disease related to tight junction dysfunction is an inflammatory disease, including but not limited to, inflammatory bowel disease (IBD), such as Morbus Crohn collagenous colitis; celiac, sprue, Crohn's disease, ulcerative colitis, multiple sclerosis; hereditary diseases, such as hereditary deafness, familial hypomagnesemia, neuroinflammation (see Grin'kina et al., PLoS ONE 7(5): e36475. doi:10.1371/journal.pone.0036475 (2012), the disclosure of which is incorporated by reference herein in its entirety), cystic fibrosis; vision loss, such as diabetic eye disease: diabetic retinopathy; viral infection, such as retroviral infection (hydrocephalus, encephalitis); bacterial toxins, such as Clostridium perfringens enterotoxin; a disease in lung tissue integrity; a disease in lymphocyte trafficking (see Mandala et al., Science 296, 346 (2002), the disclosure of which is incorporated by reference herein in its entirety, or other immunological diseases (see Rosen et al., Nature Reviews Immunology 5, 560-570 (July 2005), the disclosure of which is incorporated by reference herein in its entirety), etc. In a preferred embodiment, the present invention relates to a method of preventing or treating an inflammatory bowel disease (IBD) in a subject in need thereof, comprising administering to the subject a composition comprising an effective amount of glycerophosphate, such as calcium glycerophosphate (CGP). More preferably, the IBD is Morbus Crohn collagenous colitis or Crohn's disease, which has diarrhea as a leading symptom and is chiefly attributed to epithelial barrier dysfunction that results in an increased loss of solutes in the “leaking flux diarrhea.”

While not wishing to be bound by theories, it is believed that glycerophosphate, such as calcium glycerophosphate (CGP), elevates sphingosine-1-phosphate (S1P) concentrations and/or activity in epithelial or endothelial cells, consequently decreasing transepithelial permeability. Without wishing to be bound by theory, it is also believed that glycerophosphate salts act, at least in part, by inhibiting the action of phosphatases, including those that dephosphorylate S1P, resulting in the glycerophosphate salts mediating the formation and longevity of S1P and increasing S1P's availability to preserve junction integrity in a gut challenged by NSAIDs and exercise induced ischemia.

### Enteric Coated Compositions

The present invention also provides an enteric coated composition comprising a therapeutically effective amount of a glycerophosphate salt. According to embodiments of the present invention, an enteric coated composition according to the invention is a delayed-release formulation that can be used to treat intestinal injury, intestinal injury aggravated by an NSAID, exercise-induced intestinal injury, and exercise-induced intestinal injury further aggravated by an NSAID. Thus, embodiments of the present invention also encompass methods of treating or preventing intestinal injury or symptoms thereof in a subject comprising administering to the subject an enteric coated composition according to the invention. Embodiments of the present invention also encompass methods of increasing tight junction integrity in a subject and methods for treating or preventing a disease related to tight junction dysfunction in a subject comprising administering to the subject an enteric-coated composition according to the invention.

According to embodiments of the present invention, an enteric coated composition comprises: (i) a core comprising a therapeutically effective amount of glycerophosphate salt; and (ii) an enteric coating surrounding the core. In a preferred embodiment, the glycerophosphate salt is calcium glycerophosphate.

As used herein, the term “enteric coated composition” refers to a composition having an enteric coating that surrounds and encases the exterior of a core of the composition, wherein the core comprises the therapeutically or pharmacologically active compound. As used herein, the term “core” refers to the component of the enteric coated composition that comprises the therapeutically active ingredient. A core employed in an enteric coated composition according to the invention is preferably formulated in a solid dosage form for oral administration, such as, for example, a powder, tablet, capsule, caplet, gelcap, liquid gel, or pellet etc. Because the core of a composition according to the invention is surrounded and encased by an enteric coating, the core comprises an interior layer or portion of the composition.

As used herein, the term “enteric coating” refers to a layer that surrounds and encases the core of a composition, and is the outermost layer of the composition Preferably, the enteric coating completely surrounds and encases the core, such that the core is not exposed to any bodily fluids upon administration to a subject (e.g., saliva, gastrointestinal juices) until the enteric coating has dissolved or disintegrated. The enteric coating prevents the therapeutically or pharmacologically active ingredient in the core of the composition from being released until the enteric coating is degraded, dissolved or broken down, but permits release of the therapeutically active ingredient from the core once the enteric coating is dissolved or degraded, or is beginning to dissolve or degrade. Preferably, the enteric coating is not degraded, dissolved, or broken down, and thus does not permit release of the therapeutically active ingredient from the core until the composition reaches the intestine, and more preferably it permits the quantitative release of the therapeutically active ingredient from the core to at least the jejunum or ileum portions of the small intestine of the subject and substantially reaches the large intestine as well. In a particular embodiment, the enteric coating permits the release of the therapeutically active ingredient from the core once the composition reaches the junction between the duodenum and jejunum portions of the small intestine, such that the release of the therapeutically active ingredient begins at the jejunum portion and occurs until the composition reaches at least the distal small intestine (ileum) as well as the large intestine in effective quantity.

The enteric coating of an enteric coated composition according to the invention can be used to control the location within the gastrointestinal tract that the glycerophosphate salt is released from the core of the composition, such that the glycerophosphate salt is site-specifically delivered. As used herein, the term “site specific delivery” refers to release of the therapeutically active ingredient from the core of a composition at the intended site of delivery. According to embodiments of the present invention, an enteric coated composition site specifically delivers a glycerophosphate salt to the intestine, preferably the small intestine or large intestine, more preferably the small intestine, and most preferably the junction between the duodenum and jejunum portions of the small intestine. Thus, the enteric coating permits transition of the composition through the stomach of the subject without releasing glycerophosphate salt, and only once the composition passes into the intestinal tract and the enteric coating begins to degrade or dissolve, is the glycerophosphate salt released.

In a preferred embodiment, once the enteric coating of an enteric coated composition according to the invention has disintegrated or dissolved, substantially all of the glycerophosphate salt is released from the core of the composition at substantially the same time. In a more preferred embodiment, substantially all of the glycerophosphate salt is released from the core of the composition at the time the composition reaches the small intestine of the subject

In one embodiment, an enteric coated composition according to the invention site-specifically delivers a glycerophosphate salt to the small intestine. When site-specific delivery is to the small intestine, the delivery can be to either the duodenum, jejunum, or ileum portions of the small intestine. In another embodiment, site-specific delivery is to the large intestine. Preferably, an enteric coated composition according to the invention site-specifically delivers a glycerophosphate salt to the junction between the duodenum and jejunum portions, such that maximal release of the glycerophosphate salt does not commence until the composition reaches at least the jejunum, where there is the greatest likelihood that it will not be absorbed into the body like a nutrient, but will proceed into the ileum portion of the small intestine and large intestine to lave these sections.

According to embodiments of the present invention, the enteric coating is comprised of a polymer material. Preferably, the polymer material is pH sensitive, such that the polymer material remains intact, or is stable, at a highly acidic pH, but breaks down, dissolves, or disintegrates at a less acidic pH. A suitable pH sensitive polymer for use with the present invention is one which remains intact and is stable within the lower pH environment of the stomach (i.e., around pH 3), but will disintegrate or dissolve at a pH commonly found in the intestinal tract (i.e., around pH 5 to about pH 7).

According to embodiments of the present invention, the polymer material can be a synthetic material, such as acrylic or maleic based polymers and polyvinyl derivatives, or an organic material, such as a cellulosic based polymer. Preferably, the polymer material is pH sensitive. Examples of pH-sensitive acrylic polymers that can be used in the present invention include, but are not limited to, polymers (homopolymers or copolymers) of methacrylic acid, methyl methacrylate and ethyl acrylate, etc., such as copoly(methacrylic acid/ethyl acrylate), copoly(methacrylic acid/methyl methacrylate). A copolymer of methacrylic acid and ethyl acrylate is commercially available under the trade name Eudragit® L-30-D 55, available from Evonik Industries. Examples of pH-sensitive cellulosic polymers, that can be used in the present invention include, but are not limited to, hydroxypropylmethylcellulose phthalate (HPMCP), hydroxypropylmethylcellulose acetate succinate (HPMCAS), carboxymethylethyl cellulose (CMEC), cellulose acetate phthalate (CAP), cellulose acetate succinate (CAS), and cellulose acetate trimellitate (CAT). Other commercially available polymers and enteric coating systems that can be used with the present invention include Acryl-EZE®, Sureteric®, Nutrateric® II, and Opadry® enteric coatings all available from Colorcon, Ltd.

According to embodiments of the present invention, an enteric coating can be comprised of any polymer material, or any combination thereof, in view of the present disclosure. The polymer material can comprise about 5-90% by weight, relative to the total weight of the enteric coating, such as, for example, 10%, 20%, 30%, 40%, 50%, 60%, 70%, 80%, or 90%.

According to embodiments of the present invention, an enteric coating is stable in the acidic environment of the stomach, which has a pH of around 3, but is dissolved or degraded in a less acidic environment having a pH of about 5 or greater, such as the pH found in intestinal tract. For example, an enteric coating can be stable at a pH of about 1, 1.5, 2, 2.5, 3, 3.5 or 4, 4.5, or 5 but can be dissolved or disintegrated at a pH of about 5, 5.5, 6, 6.5, 7, 7.5, 8, 8.5 or 9.

An enteric coating according to the invention can further comprise additives, such as plasticizers, lubricants, pigments, colorants, antifoam agents, emulsifiers, surfactants, etc., to improve the properties of the enteric coating. For example, a plasticizer can increase the flexibility and elasticity, and diminish the brittleness, of the enteric coating. A plasticizer can be either a hydrophilic plasticizer or a hydrophobic plasticizer. Examples of plasticizers that can be used in an enteric coating of the present invention include, but are not limited to, phthalic derivatives, dibutylphthalate, propylene glycol, triacetine, silicone oil, diethylphthalate, triethyl citrate, dibutyl sebacate, and polyethylene glycol, etc. Examples of lubricants that can be used in an enteric coating include magnesium stearate, talc, etc.

In a preferred embodiment, an enteric coating further comprises a plasticizer. The plasticizer can comprise about 5% to about 60% by weight relative to the total weight of the enteric coating, such as about 10%, 20%, 30%, 40%, 50%, or 60%.

The pH of the intestinal tract gradually increases from a pH of about 5, found in the upper portions of the small intestine such as the duodenum, to a pH of about 7.0-8.0 in the distal portions of the small intestine, such as the ileum, and the large intestine. An enteric coated composition according to the invention is formulated such that the enteric coating will not dissolve and permit release of the glycerophosphate salt until the composition has passed through the stomach and at least reached the upper portion of the small intestine. Thus, the enteric coating can begin to dissolve within the pH range of the duodenum, and continue to dissolve at the pH range within the small intestine. The enteric coating can also be such that it does not begin to dissolve until reaching distal portions of the small intestine (i.e., ileum), or the large intestine.

Preferably, an enteric coated composition according to the invention permits the quantitative release of the glycerophosphate salt from the core as of the time the composition reaches at least the small intestine or large intestine of the subject. More preferably, an enteric coated composition according to the invention permits the quantitative release of the glycerophosphate salt from the core as of the time the composition reaches at least the jejunum portion of the small intestine.

The site within the intestinal tract in which the glycerophosphate salt will be released from an enteric coated composition according to the invention can be adjusted by varying several parameters, including the thickness of the enteric coating, identity of the polymers in the enteric coating, pH sensitivity of the enteric coating, addition of additives, etc. Preferably, the enteric coating is such that it is substantially dissolved during the transit time through the intestinal tract to provide for maximum release of the glycerophosphate salt into the intestinal tract, and preferably at the site of the jejunum or ileum.

In one embodiment, the enteric coating dissolves at a pH of about 5.5 and will site-specifically deliver the glycerophosphate salt to the duodenum portion of the small intestine. In another embodiment, the enteric coating dissolves at a pH between about 5.5 and 7.0, and will site-specifically deliver the glycerophosphate salt in either the jejunum or ileum portion of the intestine. In another embodiment, the enteric coating dissolves at a pH of about 7.0 or greater, and will site specifically-deliver the glycerophosphate salt to the large intestine. Preferably, the enteric coating dissolves at a pH between about 5.5 and 7.0.

According to embodiments of the present invention, the enteric coating can be present in a weight ratio relative to the core of between about 5% to 60%, and preferably 5% to 30%, such as, for example 10%, 25%, 20%, or 25% (e.g., the enteric coating is 5% by weight relative to total weight of the core). However, one of ordinary skill in the art will recognize that the weight ratio of the enteric coating, and the weight ratio of the composition itself, can be adjusted depending on the intended site of delivery for the glycerophosphate salt. For example, a weight ratio relative to the core of between about 5% and 30% can be appropriate for delivery to the duodenum and jejunum portions of the small intestine, but the weight ratio can be higher than 30% for delivery to the ileum portion of the small intestine, or the large intestine.

According to embodiments of the present invention, the thickness and/or technical sophistication (i.e. number of layers, type of polymers, etc.) of the enteric coating can affect the rate of dissolution of the enteric coating, and thus the location of subsequent release of the glycerophosphate salt from the core (i.e., the location of site-specific delivery). As used herein, the terms “thin” and “thick” encompass both the thickness of the coating and its technical sophistication. For example, a thin enteric coating will be dissolved or degraded more rapidly than a thick enteric coating, and release of a glycerophosphate salt from a composition comprising a thin enteric coating can occur at a location in the intestine that is more proximal to the stomach, such as the duodenum, as compared to the location in the intestine that the glycerophosphate salt is released from a composition comprising a thicker enteric coating, which will be more distal to the stomach, such as in the jejunum, ileum, or large intestine.

The thickness of the enteric coating of an enteric coated composition according to the invention can be controlled by, for example, adjusting the number of layers of the enteric coating. In one embodiment, an enteric coated composition comprises one layer of an enteric coating. In another embodiment, an enteric coated composition comprises more than one layer of an enteric coating, such as for example, 1, 2, 3, 4, or 5 or more layers. For example, increased layers of enteric coating can be used to formulate an enteric coated composition for site-specific delivery to the distal portions of the small intestine, such as the ileum, or the large intestine.

An enteric coated composition according to embodiments of the present invention can further comprise a subcoat between the enteric coating and the core. As used herein, the term “subcoat” refers to a film or coating that acts as a physical barrier between the core comprising the therapeutically active ingredient and the enteric coating surrounding the core, preventing the enteric coating from being in physical contact with the core. The subcoat is applied as a layer over the core prior to application of the enteric coating. Functions of subcoats include protection of the components of the core, such that there are no adverse reactions between the components of the core and the components of the enteric coating. For example, an enteric layer can be acidic, and direct contact of the core with an acidic enteric layer can result in destabilization or degradation of the therapeutically active component, generation of impurities, etc. A subcoat can also function to provide a smooth base over the core for even application of the enteric coating. Examples of materials suitable for use as a subcoat include, but are not limited to, cellulose polymers, such as methylcellulose, ethylcellulose, etc., acrylics, such as homo or copolymers of methacrylate and methyl methacrylate, etc., and vinyls, such as polyvinyl alcohol, etc. A subcoat can further comprise additives, such as plasticizers, lubricants, pigments, colorants, antifoam agents, emulsifiers, surfactants, etc.

An enteric coated composition according to the invention is formulated for oral administration. According to embodiments of the present invention, the core of an enteric coated composition is preferably a solid, such as, for example, a tablet, capsule, pellet, liquid gel, granule, powder, etc. Preferably, the core is in the form of a tablet. To prepare the core of an enteric coated composition, the glycerophosphate salt is mixed with a pharmaceutically acceptable carrier according to conventional pharmaceutical compounding techniques.

The core can comprise any pharmaceutically acceptable carrier in view of the present disclosure. For example, a pharmaceutically acceptable carrier can include one or more excipients such as binders, suspending agents, emulsifying agents, wetting agents, lubricants, flavorants, sweeteners, preservatives, dyes, and coatings. In particular, suitable carriers and additives for a core formulated as a solid (e.g., tablet, capsule, etc.) include starches, sugars, diluents, granulating agents, lubricants, binders, disintegrating agents and the like.

The core of an enteric coated composition for use in the present invention can comprise a therapeutically effective amount of a glycerophosphate salt that is about 10-100%, preferably 50-100%, and most preferably 75-99.9% of the total weight of the core. The amount of glycerophosphate salt will depend on the specific cation of the glycerophosphate salt. For example, if calcium glycerophosphate is used, the core preferably comprises about 99.625% calcium glycerophosphate by weight of the total core, and 0.375% by weight of a carrier(s), and preferably 0.375% by weight magnesium stearate with no other carrier(s), by weight of the total core.

According to embodiments of the present invention, the core of an enteric coated composition can further comprise an NSAID. The core can comprise an amount of the glycerophosphate salt by weight that is about 10% to about 1000% of the weight of the NSAID in the mixture of glycerophosphate salt and NSAID, preferably 30% to 70%, more preferably 40% to 60%, and most preferably 50% of the weight of the NSAID in the core. In a preferred embodiment, the NSAID is ibuprofen.

According to embodiments of the present invention, an enteric coated composition of the present invention can further comprise a controlled-release agent in at least one of the core or the enteric coating. As used herein, the term “controlled-release agent” refers to a compound or additive that controls the rate at which the therapeutically active ingredient of a composition is made available, or is tailored to possess a specific delay to govern the site at which the therapeutically active ingredient is made available to the subject to whom the composition is administered. Examples of controlled release agents include sustained release agents (for sustained release or extended release), immediate release agents (for immediate release or dispersion), and delayed release agents (for delayed release).

As used herein, the terms “sustained-release” and “extended release” refer to the release of the therapeutically active component from a pharmaceutical composition over an extended period of time. As used herein, the term “immediate release” refers the release or dispersion of the therapeutically active component from a pharmaceutical composition immediately or shortly following administration to a site in a subject. Immediate release can also refer to dispersion directly after the enteric coating of an enteric coated composition according to the invention has begun to dissolve. As used herein, the term “delayed release” refers to the release of the therapeutically active component from a pharmaceutical composition only after the composition reaches a desired locale within the subject following administration to a subject, or a specified amount of time following administration to the subject has past, such that release occurs site-specifically, or at the desired locale.

Preferably, a composition according to the invention comprises a controlled-release agent that is a delayed-release agent.

As used herein, the term “delayed-release agent” refers to a compound or additive that provides for the controlled release of the therapeutically active ingredient from a composition, such that release occurs site-specifically, or at the desired locale. A delayed-release agent can be of the type which releases substantially all of the therapeutically active ingredient at the same time, once it reaches the desired locale, or releases the therapeutically active ingredient only after a specified amount of time following administration of the composition to the subject has past, such that the composition reaches a desired locale, and the therapeutically active ingredient is thus delivered to the desired locale, or site-specifically. According to embodiments of the present invention, the delayed-release agent can be present in the composition as part of the core, or as part of the enteric coating. Preferably, the delayed-agent is present as part of the enteric coating.

According to embodiments of the present invention, an enteric coated composition further comprising a delayed-release agent allows for the release of the glycerophosphate salt from the core only once the composition has at least entered the small intestine or large intestine, and preferably has reached the junction between the duodenum and jejunum portions of the small intestine. Preferably, release of the glycerophosphate salt from the core of the composition can occur over a governing pH range that is at least greater than 5.0, such as 5.5, 6.0, 6.5, 7.0, 7.5, or 8.0 or greater, and preferably occurs at a pH that is at least 7.0 or greater.

The amount of the delayed-release agent present in a composition according to the invention will depend upon the desired release profile of the glycerophosphate salt. For example, the delayed-release agent can be present in a percentage by weight that is between about 10% and 90% by weight of the total weight of the composition, such as 10%, 20%, 30%, 40%, 50%, 60%, 70%, 80%, or 90%. When the delayed-release agent has a release profile that is particularly pH durable (i.e., does not dissolve until higher pHs, such as at least pH 7.0), the delayed-release agent can be present in a percentage by weight of the total weight of the composition that can be smaller, such as 10%, 20%, 30%, 40%, or 50%.

Examples of delayed-release agents suitable for use in the present invention include, but are not limited to, gels, waxes, fats, emulsifiers, combinations of fats and emulsifiers, polymers, starch, cellulose polymers, etc. and combinations thereof. Examples of waxes and waxy materials that can be used to provide delayed-release include canuba wax, spermaceti wax, candellila wax, cocoa butter, cetosteryl alcohol, beeswax, partially hydrogenated vegetable oil, cerasin, paraffin, myristyl alcohol, stearyl alcohol, cetyl alcohol, and stearic acid.

Preferably, a delayed-release agent comprises a rate-controlling polymer. As used herein, the term “rate-controlling polymer” refers to a polymer that controls the rate at which a therapeutically active ingredient is released from a composition, such that the therapeutically active ingredient is not released until the composition has reached a desired locale, or intended site of delivery. Examples of rate controlling polymers include cellulose polymers, such as hydroxypropylmethylcellulose (HPMC), hydroxyethylcellulose (HEC), hydroxypropylcellulose (HPC), carboxy methyl cellulose (CMC), and mixtures thereof. According to embodiments of the present invention, the molecular weight of a cellulose polymer to be used as a delayed-release agent can be varied to optimize the rate of controlled-release of the glycerophosphate from the core in order to achieve the desired release profile.

There are several commercially available rate-controlling polymers suitable for use in the present invention. These include METHOCEL® Cellulose Ethers and ETHOCEL® Ethylcellulose polymers, available from Colorcon, Ltd.

According to a preferred embodiment of the present invention, a rate-controlling polymer is a pH sensitive polymer that is present in a composition according to the invention as part of the enteric coating. In this embodiment, the enteric coating itself provides delayed-release properties to the composition, such that the enteric coating does not dissolve and allow for release of the glycerophosphate salt from the core until the composition reaches a site of the intestinal tract that has a pH at which the pH sensitive polymer will dissolve, degrade, or disintegrate.

Methods are known in the art for forming enteric coated compositions, and particularly for forming enteric coated compositions wherein the composition is in the form of a solid for oral administration. An enteric coated composition for use in a method of the present invention can be produced by applying either a solution or suspension of the enteric coating to a core comprising a therapeutically effective amount of a glycerophosphate salt, wherein the core is preferably in a solid oral dosage form, such as a tablet, capsule, pellet, liquid gel, granule, powder, etc., and is more preferably a tablet.

According to embodiments of the present invention, a coating formulation for application to the core is first produced. As used herein, the term “coating formulation” refers to an enteric coating according to the invention that is in the form of a suspension/dispersion or a solution. Thus, the coating formulation comprises all the components of the enteric coating either suspended or dissolved in a solvent (organic or aqueous). The coating formulation can be either an aqueous suspension or dispersion, or it can be an organic solution.

A coating formulation in the form of an aqueous suspension or dispersion can be prepared by diluting or suspending a polymer material and any additives, such as lubricants, plasticizers, delayed-release agents etc., in an aqueous solvent, and preferably in water. A coating formulation in the form of a solution can be prepared by dispersing or dissolving a polymer material and any additives in an organic solvent. Examples of suitable solvents that can be used include acetone, methanol, ethanol, isopropyl alcohol, ethyl acetate, methylene chloride or mixtures thereof, etc. Preferably, a plasticizer is added to the coating formulation, whether it is in the form of an aqueous suspension/dispersion or solution.

Any method known in the art for forming suspensions and solutions can be used in view of the present disclosure, including, but not limited to, mixing, blending, stirring, etc.

The coating formulation is then applied to the core to obtain an enteric coated composition according to the invention. Any method known in the art for applying the coating formulation to the core to produce an enteric coated composition according to the invention can be used. Typical coating methods for applying a coating formulation include spray coating methods such as fluidized bed processing, spray drying, and side vented pan coating processes. Examples of machines that can be used to carry out spray coating methods include a fluidized bed coating machine, a centrifugal fluidized bed coating machine, a pan coating machine, and the like. Generally, such processes involve applying a coating formulation, as described above, by spraying the coating formulation onto the core using a spray nozzle, followed by drying to allow evaporation of the solvent originally present in the coating formulation. The temperature of the drying step should be in the range advised by the manufacturer of the particular enteric coating being used.

According to embodiments of the present invention, a coating formulation is applied to the core at least once, but can be applied to the core more than once to obtain an enteric coated composition with one or more layers of enteric coating. In certain embodiments, it can be advantageous to apply more than one layer of enteric coating, wherein each layer comprises a different polymer material, or additive component, in order to optimize the dissolution properties, or release profile, of the glycerophosphate salt from the enteric coated composition.

A subcoat can also be applied to the core prior to application of the enteric coating. Techniques are known in the art for applying subcoats to solid pharmaceutical compositions for oral administration. Such techniques include spraying the subcoat onto particles, such as tablets, capsules, etc., present in a rotating pan, using a fluidized bed coating apparatus, and the like.

The release profile of the glycerophosphate salt from the core of the enteric coated composition will be affected by a variety of parameters including the thickness of the enteric coating, additives in the coating, the solubility of the glycerophosphate salt, and the pH sensitivity of the polymer material. One of ordinary skill in the art will be able to readily optimize these parameters in order to obtain an enteric coated composition for use in a method of the present invention with the desired dissolution or release profile.

### Methods of Treatment Using Enteric Coated Compositions

Enteric coated compositions comprising a therapeutically effective amount of a glycerophosphate salt, preferably calcium glycerophosphate, can be used in any method for treating, reducing, alleviating, or preventing intestinal injury or symptoms thereof in a subject in view of the present disclosure.

According to embodiments of the present invention, the intestinal injury to be treated or prevented can be aggravated by an NSAID (preferably ibuprofen), exercise-induced, or exercise-induced and further aggravated by ingestion of an NSAID. Preferably, the intestinal injury to be treated or prevented is aggravated by an NSAID. For example, enteric coated compositions according to the invention can be used in a method for treating or preventing intestinal injury or symptoms thereof aggravated by an NSAID in a subject, for treating or preventing exercise-induced intestinal injury or symptoms thereof in a subject, or for treating or preventing exercise-induced intestinal injury or symptoms thereof further aggravated by an NSAID in a subject.

Preferably an enteric coated composition is orally administered to a subject. Preferably, the glycerophosphate salt is calcium glycerophosphate.

In a particularly preferred embodiment, the present invention provides a method of treating or preventing intestinal injury or symptoms thereof, including those which are exercise-induced, aggravated by an NSAID in a subject, wherein the subject is ingesting an NSAID, comprising orally administering to the subject an enteric coated, composition according to the invention, wherein the glycerophosphate salt present in the core of the composition is calcium glycerophosphate. In a preferred embodiment, the NSAID is ibuprofen.

In another particularly preferred embodiment, the method comprises administering to the subject an enteric coated composition having a core comprising an effective amount of glycerophosphate salt, preferably calcium glycerophosphate, and an effective amount of NSAID, preferably ibuprofen, such that the NSAID and calcium glycerophosphate, are co-administered as part of the same composition.

In another particularly preferred embodiment, the enteric coated composition further comprises a delayed release agent.

Enteric coated compositions comprising a therapeutically effective amount of a glycerophosphate salt, preferably calcium glycerophosphate, can be used in any method for increasing tight junction integrity in a subject in need thereof and for treating or preventing a disease related to tight junction dysfunction as previously described in a subject in need thereof in view of the present disclosure.

It is believed that the functioning moiety of the glycerophosphate salt is the glycerophosphate component. Although calcium is the preferred cation for the glycerophosphate salt, nonetheless, the presence of calcium along with the glycerophosphate component is not essential to the activity of glycerophosphate in this arena and other appropriate non-toxic cations can be used in the present invention including, but not limited to, glycerophosphate salts of sodium, potassium, and magnesium. However, CGP is the preferred glycerophosphate salt because a loosely bound calcium ion is present in CGP which breaks away from the glycerophosphate moiety immediately upon encountering moisture, resulting in the instantaneous availability of both ions in an aqueous milieu, and which, while present in minimal quantities relative to already existing calcium, still is implicated in its reinforcement of the effect of glycerophosphate on the engendering and longevity of SIP. Further, to the degree that the normal E-cadherins are functioning, calcium in any amount is beneficial; the very name “cadherin” is derived from “calcium-dependent adhesion.” It should be emphasized, however, that the CGP effect is not the consequence of added calcium, since the media already contains 1 mM calcium, and there are effects demonstrated at 1 μM GCP.

The use of CGP and other glycerophosphate salts for additional purposes not disclosed in the present application has been described in various other patents, both pending and granted. For example, U.S. Pat. No. 5,869,119 describes the use of CGP in increasing the pH of acidic foods and beverages to reduce heartburn and gastrointestinal distress, and U.S. Pat. No. 7,402,323 describes the beneficial effects of CGP in cellular repair. While not wishing to be bound by theory, it is believed that the beneficial effects of CGP in various applications are achieved synergistically between the calcium ion and the glycerophosphate moiety present in the CGP molecule. In preventing and/or treating exercise-induced intestinal injury and exercise-induced intestinal injury aggravated by ingestion of an NSAID according to the present invention, CGP may be instrumental in, among other benefits, fomenting tight junctions among intestinal cells. For this reason, CGP is effective at increasing tight junction integrity and preventing or treating diseases related to tight junction dysfunction.

This invention will be better understood by reference to the non-limiting examples that follow, but those skilled in the art will readily appreciate that the examples are only illustrative of the invention as described more fully in the claims which follow thereafter.

### EXAMPLES

Experiments were conducted on Caco-2 cells, a line of heterogeneous human epithelial colorectal adenocarcinoma cells derived from a colorectal carcinoma and grown on transwell permeable inserts. Despite their origins, in culture these cells express the characteristics of small intestinal enterocytes upon prolonged culture (C. Jumarie et al. J Cell. Physiol. 149: 24-33, (1991)). They are widely used as a model of transepithelial transport in the small intestine, and are considered a model for celiac disease (G. Iacomino et al. J Agric Food Chem. 6; 61(5):1088-962013 (2013); I. Caputo et al. PLoS One. 7(9):e45209 (2012); T. Rauhavirta et al. J Clin Immunol.; 33(1):134-42 (2013)).

(1) Measurement of Transepithelial Electrical Resistance:

The tightness of the tight junction in a transporting epithelium can be evaluated by measuring the electrical resistance across the membrane or trans-epithelial electrical resistance (TEER). TEER measures the movement of ions across the cell (transcellular) and between cells (paracellular). In a very tight monolayer, transport is nearly all transcellular, hence the electrical resistance is high. Cells are grown on a permeable barrier (e.g., the transwell system), and the electrical resistance across the membrane is measured using an ohmmeter (e.g., the EVOM2 epithelial voltohmmeter) specifically designed for the purpose. At 3 to 4 weeks in culture, the TEER may be in the range of 700-800 Ω/cm².

(2) Mannitol Flux:

The TEER results are verified by measuring the flux of mannitol, a polyhydroxy alcohol, across the cell monolayer. Mannitol flux is exclusively by the paracellular route, so that its movement is inversely proportional to the tightness of the tight junctions. Flux is measured in both apical to basolateral and basolateral to apical directions. The rate of flux is used to calculate a permeability coefficient for the monolayers. The permeability coefficient for mannitol may be inversely related to TEER.

(3) E-Cadherin Expression:

E-cadherin is the principle protein of tight junctions. The level of E-cadherin is measured in homogenized Caco-2 cells by western blot analysis using commercially available antibodies. Since it is possible that E-cadherin might be expressed as a protein, but sequestered away from the cell membrane, surface E-cadherin is also measured using either flow cytometry or fluorescent microscopy. The ratio of surface to total E-cadherin is calculated. The expression levels of E-cadherin are measured in Caco-2 cells in the presence and absence of CGP. The expression levels of other proteins of tight junctions in Caco-2 cells in the presence or absence of CGP can also be measured using methods known in the art in view of the present disclosure.

(4) Sphingosine-1-Phosphate:

Calcium glycerophosphate has activity as a phosphatase inhibitor. It is believed that CGP elevates S1P concentrations by inhibiting dephosphorylation. S1P concentrations are measured in the media of Caco-2 cells using a commercially available ELISA kit (e.g., Echelon Catalog # K-1900). The S1P concentrations are measured in the media of Caco-2 cells in the presence and absence of CGP.

### Experimental Conditions

Baseline values were obtained for each of these parameters in unstimulated cells in the presence and absence of increasing concentrations of CGP. The time course of TEER development, as well as the appropriate concentration of CGP, was determined experimentally. The TEER development in Caco-2 cells takes from 18 to 24 days after the monolayers reach confluence, while the CGP concentrations are anticipated to range from 1 μM to 1 mM. The mannitol flux and TEER data are used to differentiate transcellular and paracellular permeabilities. At baseline, paracellular permeability can be very low relative to transcellular permeability, reflecting the “tightness” of the tight junctions.

The effect of hypoxia on transepithelial permeability was measured for each parameter in the presence and absence of CGP. Cells were grown to confluence in normoxic conditions on transwell inserts, then transferred to a hypoxia chamber (95% N₂/5% CO₂) and media was changed to one sparged with 95% N₂/5% CO₂ and containing no glucose. Under these conditions, which simulate ischemia, the partial pressure of residual oxygen is less than 25 mm Hg (6%). Control wells contained only media; the remaining wells contained increasing concentrations of calcium glycerophosphate. TEER and mannitol flux were measured in the hypoxia chamber. E-Cadherin expression or S1P determination was determined in room air using samples prepared in the hypoxia chamber. Hypoxia may decrease TEER and S1P, increase both apical to basolateral and basolateral to apical mannitol flux without changing E-cadherin expression. These effects may be mitigated by CGP.

The effect of exogenous cytokines was measured for each parameter in the presence and absence of CGP. Both hypoxia and celiac disease are characterized by the release of cytokines by cells of the immune system. These inflammatory mediators increase monolayer permeability (M. Bayardo et al. Clin Exp Immunol.; 168(1):95-104 (2012)). Cells were grown to confluence on transwell inserts, then challenged with a mixture of the cytokines TNFα (tumor necrosis factor alpha), ILβ (interleukin beta), and IFγ (interferon gamma) (“cytomix”) in the presence or absence of increasing concentrations of CGP. At the appropriate time points, apical to basolateral mannitol flux was measured. TEER, E-Cadherin expression and media concentrations of S1P were measured in separate experiments. In addition, the expression of iNOS was measured to verify that the cytokine treatment resulted in an inflammatory response. Exogenous cytokines may decrease TEER and increase mannitol flux.

The effect of α-gliadin peptides is measured for each parameter. α-Gliadin is the offending protein initiating the symptoms of celiac disease in susceptible patients. Gastric and pancreatic proteases cleave α-gliadin to peptides, which may be the direct cause of the enteropathy. The literature (G. Iacomino et al. J Agric Food Chem. 6; 61(5):1088-962013 (2013)) suggests that the peptide comprised of α-gliadin amino acids 31-55 is a likely candidate. A most likely candidate (consensus of other investigators) was selected and each parameter in these experimental conditions was measured, as shown in Table 1:

TABLE 1

| Peptide 31-55 | CGP | Cytokine |
|---|---|---|
| Present | Absent | Absent |
| Present | Present | Absent |
| Present | Present | Present |

The peptides may increase permeability, and may act synergistically with the cytokines. CGP may return permeability toward normal.

Mannitol Flux Assay was performed as follows. At the start of the experiment (time 0), the media on the cells was changed to one containing no glucose and no fetal calf serum, and the environment was changed from 95% oxygen/5% CO₂, to 1% oxygen, 5% CO₂ and 94% nitrogen. These conditions mimic ischemia (no food, no oxygen).

### Experimental Results and Discussion

Baseline: Mannitol Flux.

The effect of CGP on baseline mannitol flux measured in Caco-2 cells grown on transwell permeable inserts is shown in FIG. 1. There was no significant effect of CGP on mannitol flux across the barrier. However, it is noted that at each of the time points, the mannitol permeability was greatest in the untreated cells.

Baseline: TEER.

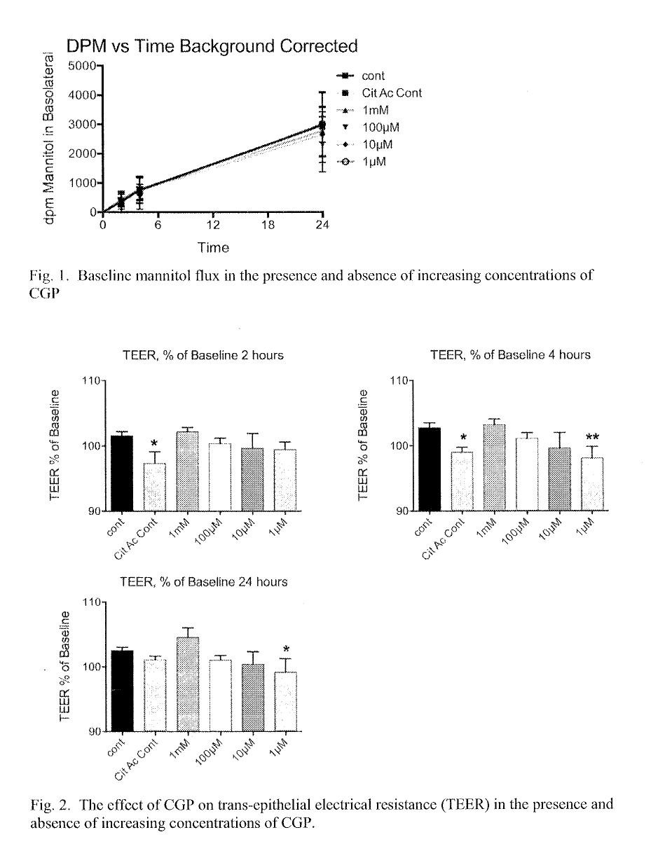

The effect of CGP on baseline TEER is shown in FIG. 2. It may be seen that citrate produces a small but statistically significant reduction in TEER, particularly noticeable at two hours. This is prevented by CGP, although the CGP effect is gone by four hours.

Baseline: S1P.

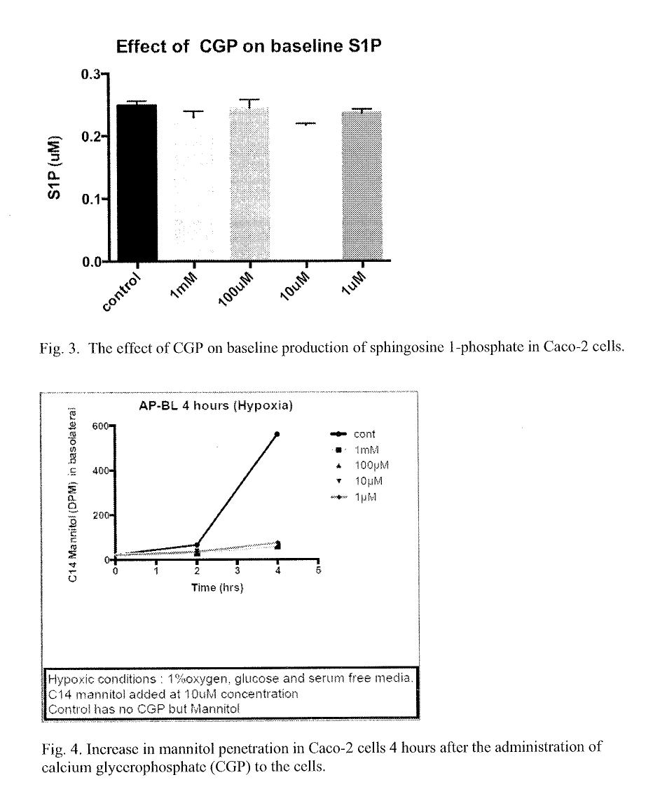

The effect of CGP on sphingosine 1-phosphate concentrations in the media is shown in FIG. 3. Cells were incubated for 24 hours in the presence of increasing concentrations of CGP. The media was removed and assayed for CGP by enzyme-linked immunoassay (ELISA). It may be seen that under baseline conditions, CGP does not appear to have any effect on sphingosine 1-phosphate concentrations.

Baseline: E-Cadherin.

It was observed that CGP may increase, but most likely does not change, E-Cadherin expression.

Altogether, the data show that CGP has only a small effect on transepithelial permeability under baseline conditions.

Mannitol Flux Assay.

It was observed that cells responded to the ischemia conditions by letting go of non-essential functions, like maintaining tight junctions. It appears that by adding CGP, the cells were able to retain tight junction integrity for a longer period of time.

Specifically, Table 2 and FIG. 4 show the percentage increase in mannitol penetration at two and four hours after CGP administration. The movement of mannitol is inversely proportional to the tightness of the tight junctions. It can be seen that the magnitude of the increase in tightness of the tight junctions (or decrease in mannitol flux) was proportional to the concentration of CGP in the media. While it is possible that the cells are merely using the glycerol phosphate to replace glucose, this is extremely unlikely, as the effect persists even at 0.000001 molar CGP. The usual glucose concentration in the media is around 10 mM, ten times greater than the highest CGP concentration, and 10,000 times greater than the lowest concentration of CGP. Thus, the results in Table 2 and FIG. 4 indicate that administering CGP to the cell resulted in increased tight junction integrity for a longer period of time.

TABLE 2

Disintegrations Per Minute

| CGP Concentration (M) | 2 hours | 4 hours |
|---|---|---|
| 0 | 188.6054 | 2362.6050 |
| 0.001 | 23.10722 | 150.2845 |
| 0.0001 | 50.46854 | 188.7996 |
| 0.00001 | 67.95316 | 195.9237 |
| 0.000001 | 77.03592 | 275.0632 |

### Transepithelial Permeability Under Conditions of Hypoxia

Hypoxia/ischemia is a state of diminished oxygen and nutrient supply such that cellular demands are not met. It was previously observed that heavy exercise, such as long-distance cycling, induces a state of increased transepithelial permeability that is exacerbated by non-steroidal anti-inflammatory agents such as ibuprofen. It was hypothesized that the increased transepithelial permeability is the consequence of intestinal hypoxia (itself a consequence of diversion of blood flow from the gut during heavy exercise), and that the permeability might be alleviated by CGP treatment. The hypothesis was tested by placing Caco-2 cells in a hypoxia chamber and measuring both apical to basolateral mannitol flux and TEER.

Mannitol Flux.

FIG. 5 shows the effect of CGP on mannitol flux during hypoxia. For these studies, [¹⁴C]mannitol was added to the apical chamber of the transwell, and sampling was from the basolateral chamber. CGP significantly reduced hypoxia induced mannitol flux in a concentration dependent manner. The effect is time dependent, as it was diminished by four hours and absent by five hours.

TEER.

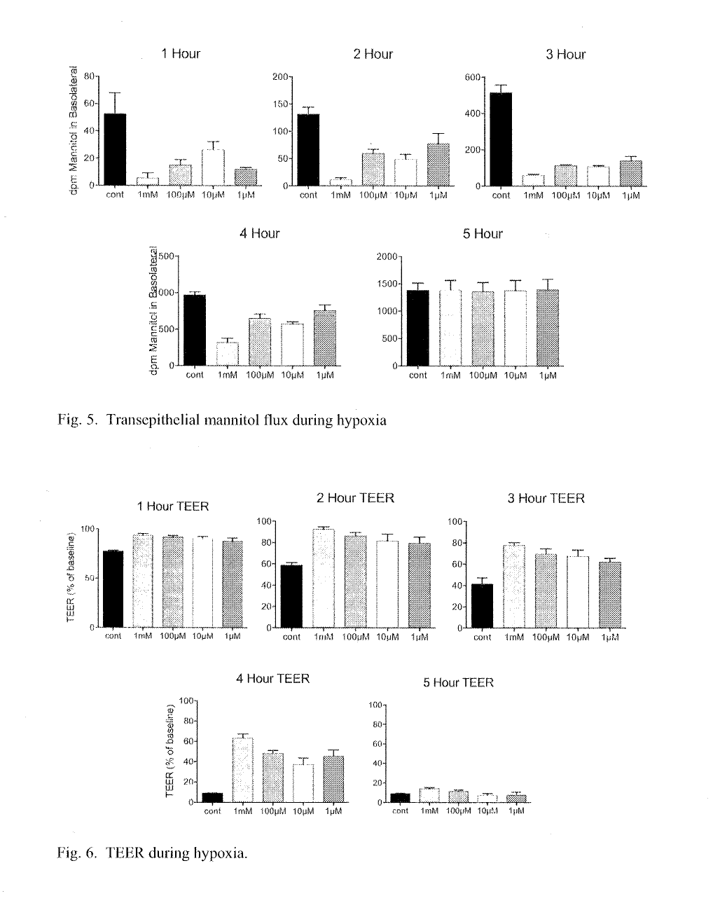

FIG. 6 shows the effect of CGP on TEER during hypoxia. For these studies, cells were grown on transwells until confluent, as measured by TEER values of >600 mΩ/cm². As there is some variability in baseline TEER, all data have been expressed as a % of baseline TEER. After 1 hour hypoxia, there was a significant decrease in TEER in the control cells. The control TEER continued to drop for the duration of the experiment. However, CGP preserved TEER in a concentration dependent manner. As for mannitol, the effect of CGP is diminished with time, so that by five hours there was no longer a discernible effect.

S1P and E-Cadherin.

(Theoretical Example) It is hypothesized that the effect of CGP is mediated through S1P. The hypothesis is tested by placing cells in the hypoxia chamber in the presence or absence of CGP. At two and four hours, samples of the media are removed and assayed for S-1P and for E-cadherin (the major tight junction protein). It is expected that S-1P will be greater in the CGP treated cells, and that there will be no change in the E-cadherin. These results will indicate that the preservation of transepithelial integrity is mediated via S1P, rather than via E-cadherin.

### Transepithelial Permeability Following Cytokine Stimulation

Cytokines are a category of small proteins that are important in cell-cell signaling. There are many types and functions of cytokines. Three of the cytokines, TNFα (tumor necrosis factor alpha), ILβ (interleukin beta), and IFγ (interferon gamma) are involved in cell signaling during inflammation. Hence, treating cells with a combination of TNF-α, IF-γ, and IL-β mimics the early phases of the inflammatory response Inflammation is relevant to the present study for two reasons. First, neutrophils and other cells mediating the immune response are recruited to ischemic tissue. Second, the intestinal damage of celiac disease is dependent on inflammation. It was hypothesized that cytokine stimulation will increase transepithelial permeability in Caco-2 cells, and that the effect will be reduced by concurrent treatment with CGP.

Mannitol Flux.

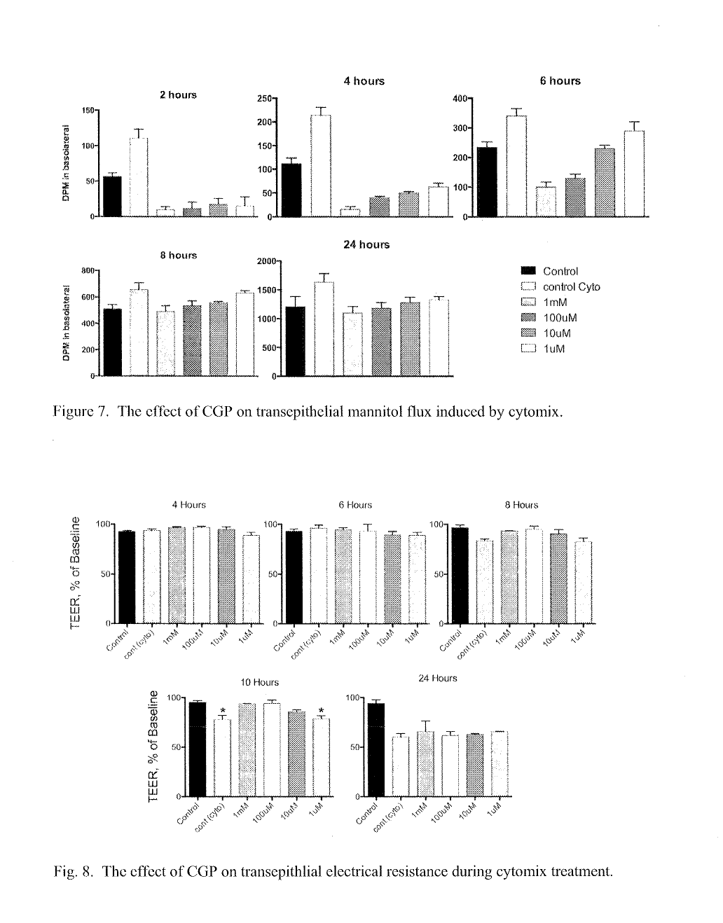

As shown in FIG. 7, cytomix alone increased transepithelial mannitol permeability in a time-dependent fashion, and CGP delayed the permeability increase in a concentration-dependent manner. Interestingly, in these experiments, control mannitol permeability was significantly greater than in the CGP/cytomix treated cells. This is in contrast to the baseline studies, in which CGP alone appeared to have very little, if any, influence on mannitol flux.

TEER.

The results of the cytomix/TEER experiment were quite different and unexpected. As shown in FIG. 8, it is clear that cytomix does not begin to reduce TEER until eight hours exposure. At ten hours, all CGP treated groups except 1 μM has a TEER significantly greater than cytomix alone and not different from control. By 24 hours, all CGP effect was lost. These results are in marked contrast to mannitol flux, and may suggest different molecular mechanisms for mannitol flux and TEER maintenance. The results indicate that additional experiments at time points between eight and ten hours would be helpful.

S1P and E-Cadherin.

(Theoretical Example) It is hypothesized that the effect of CGP is mediated through S-1P. The hypothesis is tested by treating cells with cytomix in the presence or absence of CGP. At 2 hours and 4 hours, samples of the media are removed and assayed for S-1P and for E-cadherin (the major tight junction protein).

Effect of α-Gliadin Peptides (Theoretical Example)

The α-gliadin peptide fragment 31-55 is the offending peptide initiating the symptoms of celiac disease in susceptible patients. Mannitol flux and TEER are measured for the following experimental settings:

1. Peptide only
2. Peptide plus 1 mM CGP
3. Peptide plus cytomix
4. Peptide plus cytomix plus 1 mM CGP.

At the end of the TEER experiments, the media is assayed for S-1P and E-cadherin and the cells themselves are processed for E-cadherin.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method of treating or preventing an intestinal injury or symptom thereof in a subject, the method comprising administering a composition comprising a therapeutically effective amount of a glycerophosphate salt to the subject such that the intestinal injury or symptom thereof is treated or prevented.

2. The method of claim 1, wherein the glycerophosphate salt is calcium glycerophosphate.

3. The method of claim 1, wherein the intestinal injury or symptom thereof is exercise-induced, and wherein the subject engages in exercise.

4. The method of claim 1, wherein the composition is an enteric-coated composition comprising:
(i) a core comprising the therapeutically effective amount of the glycerophosphate salt; and
(ii) an enteric coating surrounding the core.

5. The method of claim 4, wherein the enteric coating prevents release of the glycerophosphate salt from the core until the composition has reached at least the small intestine or large intestine of the subject, such that the intestinal injury or symptom thereof is treated or prevented.

6. The method of claim 4, wherein the intestinal injury or symptom thereof is exercise-induced, and wherein the subject engages in exercise.

7. The method of claim 4, wherein at least one of the core and the enteric coating of the composition further comprises a delayed-release agent.

8. The method of claim 4, wherein the core further comprises a nonsteroidal anti-inflammatory drug (NSAID).

9. The method of claim 1, wherein the composition is administered orally or nasally.

10. A method of treating or preventing an intestinal injury or symptom thereof aggravated by a nonsteroidal anti-inflammatory drug (NSAID) in a subject, wherein the subject is administered the NSAID, the method comprising administering a composition comprising a therapeutically effective amount of a glycerophosphate salt to the subject such that the intestinal injury or symptom thereof aggravated by the NSAID is treated or prevented.

11. The method of claim 10, wherein the glycerophosphate salt is calcium glycerophosphate.

12. The method of claim 10, wherein the composition comprises the therapeutically effective amount of the glycerophosphate salt and the NSAID.

13. The method of claim 10, wherein the intestinal injury or symptom thereof is exercise-induced, and wherein the subject engages in exercise.

14. The method of claim 10, wherein the composition is an enteric-coated composition comprising:
(i) a core comprising the therapeutically effective amount of the glycerophosphate salt; and
(ii) an enteric coating surrounding the core.

15. The method of claim 14, wherein the enteric coating prevents release of the glycerophosphate salt from the core until the composition has reached at least the small intestine or large intestine of the subject, such that the intestinal injury or symptom thereof is treated or prevented.

16. The method of claim 14, wherein the intestinal injury or symptom thereof is exercise-induced, and wherein the subject engages in exercise.

17. The method of claim 14, wherein at least one of the core and the enteric coating of the composition further comprises a delayed-release agent.

18. The method of claim 14, wherein the core further comprises a nonsteroidal anti-inflammatory drug (NSAID).

19. The method of claim 10, wherein the composition is administered orally or nasally.

20. An enteric coated composition comprising:
(i) a core comprising a therapeutically effective amount of a glycerophosphate salt; and
(ii) an enteric coating surrounding the core.

21. The composition according to claim 20, wherein the glycerophosphate salt is calcium glycerophosphate.

22. The composition of claim 20, wherein the enteric coating prevents release of the glycerophosphate salt from the core until the composition has reached at least the small intestine or large intestine of the subject.

23. The composition according to claim 20, wherein the core further comprises a non-steroidal anti-inflammatory drug (NSAID).

24. The composition according to claim 20, wherein at least one of the core and the enteric coating further comprises a delayed-release agent.

25. A method of increasing tight junction integrity in a subject in need thereof, comprising administering to the subject a composition comprising an effective amount of glycerophosphate.

26. The method of claim 25, wherein the composition is an enteric-coated composition comprising:
(i) a core comprising the therapeutically effective amount of the glycerophosphate salt; and
(ii) an enteric coating surrounding the core.

27. A method of treating or preventing a disease related to tight junction dysfunction in a subject in need thereof, comprising administering to the subject a composition comprising an effective amount of glycerophosphate.

28. The method of claim 27, wherein the disease is selected from the group consisting of an inflammatory disease and a cancer.

29. The method of claim 28, wherein the inflammatory disease is selected from the group consisting of inflammatory bowel disease (IBD), Crohn's disease, celiac, sprue, ulcerative colitis, multiple sclerosis, a hereditary disease, vision loss, a viral infection, bacterial toxins, neuroinflammation, a disease in lung tissue integrity, a disease in lymphocyte trafficking, and another immunological disease.

30. The method of claim 28, wherein the glycerophosphate is calcium glycerophosphate.

31. The method of claim 28, wherein the composition is an enteric-coated composition comprising:
(i) a core comprising the therapeutically effective amount of the glycerophosphate salt; and
(ii) an enteric coating surrounding the core.
